(19) United States
(12) Patent Application Publication
(10) Pub. No.: US 2025/0099302 A1
(43) Pub. Date: Mar. 27, 2025

(54) Hinged Window Bandage

(71) Applicant: James Pliny Whitney, III, Nokomis, FL (US)

(72) Inventor: James Pliny Whitney, III, Nokomis, FL (US)

(21) Appl. No.: 18/474,885

(22) Filed: Sep. 26, 2023

(51) Int. Cl. A61F 13/02; A61F 13/00

(52) U.S. Cl. CPC A61F 13/0233; A61F 13/00059; A61F 2013/0057; A61F 2013/00604; A61F 2013/00859

(57) ABSTRACT

A hinged window bandage is presented having a base border layer open in the center to allow exposure and access to a patient's wound site and a top dressing layer. The base border layer adheres to the patient's skin on its bottom-facing side, and its upper-facing side has measurement markings printed along the edge of the open window. One end of the base border is of a rougher texture than the remaining surface area. The rougher-textured end provides stronger adhesion to the top dressing layer. The top dressing layer has a wound contact dressing and lift tabs. When the top dressing layer is lifted by the lift tabs it remains adhered to the rougher textured end of the base border layer, opening in a hinged-like manner. Additional force can be used to lift the top dressing layer completely should it need to be changed.

### FIELD OF INVENTION

The present invention relates to the field of medical wound care, specifically to a hinged window bandage.

### BACKGROUND OF THE INVENTION

Certain wounds require constant monitoring at frequent intervals for signs of infection, drainage, progression in size of the wound, or other indications that the wound needs immediate medical attention. This normally involves either peeling a bandage back from the patient's skin to access the wound site or utilizing a bandage with a clear window over the wound for the healthcare worker to visually observe, document and treat the wound site. Many patients with such serious wounds requiring observation tend to have skin sensitivities or breakdown that lead to irritations and secondary injuries like skin abrasions, tears and rashes, so the process of peeling back a bandage away from the skin numerous times a day can be both tedious, time consuming for medical personnel, and excruciatingly painful for the patient. Repeating lifting of the edges of the bandage also degrades the effectiveness of the adhesive over time.

The present invention addresses a long felt need in the art by presenting a hinged window bandage having a hinged element providing the ability to view and access the patient's wound site without having to peel back the bandage completely away from the patient's skin. The improvements in function, performance, and prevention of secondary injuries to the patient make the present invention unique, useful, and highly marketable over all prior art in the field.

A first object of the invention is to provide a window bandage comprised of multiple layers having a hinged flap that can be easily opened to allow access to the wound without having to peel the bandage away from the patient's skin.

A second object of the invention is to provide a hinged window bandage having a base border layer so that wound dressings can be changed without the need to completely remove the bandage.

A third object of the invention is to provide a hinged window bandage having a top dressing layer that can be opened in a hinged manner to expose the wound for observation, documentation, and treatment without risking further injury to the patient's skin.

The present invention fulfills the above and other objects by providing a hinged window bandage having a base border layer that is open in the center to allow exposure and access to the patient's wound site. The base border layer adheres to the patient's skin on its bottom-facing side, and its upper-facing side has measurement markings printed along the edge of the open window for convenient measuring of the patient's wound. One end of the base border layer is of a rougher texture while the remaining surface area is smoother. The rougher textured end provides stronger adhesion to a top dressing layer than the remaining surface area of the base border layer. The top dressing layer has an adhesive border facing the base border layer, a wound contact dressing, and lift tabs located on the end opposite a hinged end. Any type of dressing known in the art can be manufactured and used in the top dressing layer. When the top dressing layer is lifted by the lift tabs, it remains adhered to the rougher textured end of the base border layer and opens in a hinge-like manner to expose the wound site. Additional force can be used to lift the top dressing layer completely should it need to be changed.

The above and other objects, features and advantages of the present invention should become more readily apparent to those skilled in the art upon a reading of the following detailed description in conjunction with the drawings, wherein there is shown and described illustrative embodiments of the invention.

### BRIEF DESCRIPTION OF THE DRAWINGS

In the following detailed description, reference will be made to the attached drawings in which:

FIG. 1 shows the preferred embodiment of the hinged window bandage disclosed by the present invention in a closed state.

FIG. 2 shows the preferred embodiment of the hinged window bandage disclosed by the present invention where the top dressing layer is in an open state exposing the wound site. The top dressing layer is lifted and remains adhered to the bottom border layer in a hinge-type manner.

FIG. 3 shows the top dressing layer being removed completely, allowing it to be replaced with a new top layer as needed.

FIG. 4 shows a second embodiment of the invention with alternate placement of the lift tabs on one corner of the bandage opposite to the hinged end of the bandage.

FIG. 5 shows an additional embodiment of the invention with alternate placement of the lift tabs on the center of the side opposite to the hinged end of the bandage.

### DESCRIPTION OF THE PREFERRED EMBODIMENT OF THE INVENTION

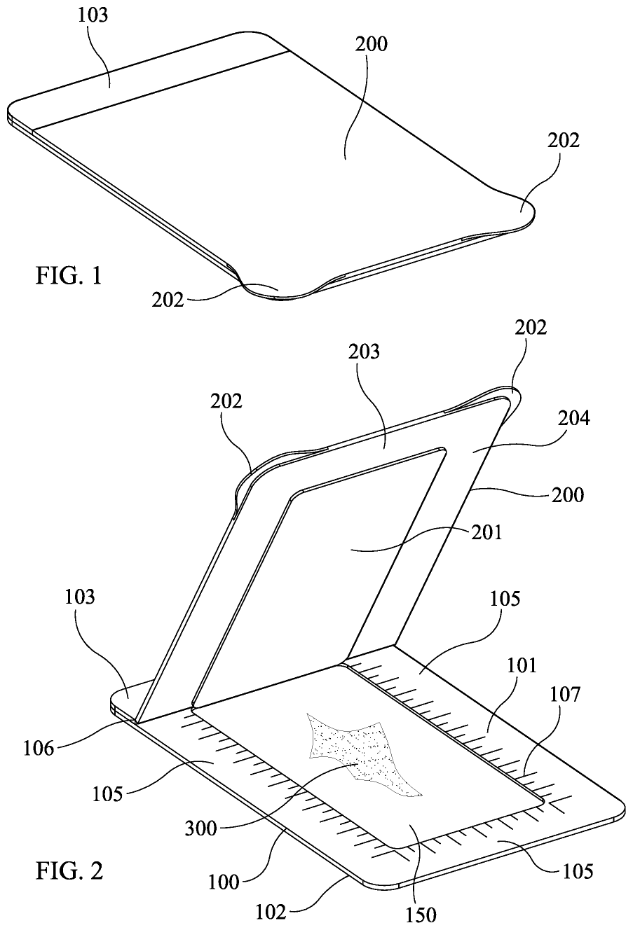

The invention has a base border layer 100 and a top dressing layer 200. The invention can be made in various sizes and shapes designed to be big enough to surround a wound. FIGS. 1—show a general rectangular shape, but the bandage can also be made in other shapes such as circles or wing shapes commonly used in the medical field for wound care. The base border layer 100 has a bottom surface 102 with a non-abrasive adhesive to affix the dressing to the skin surrounding the wound site 300. Measurement markings 107 are printed on the base border layer 100 to conveniently measure wound size, especially where wound photographs are commonly taken, eliminating the need for medical personnel to hold a ruler while taking a photograph at the same time. One end of this base border layer 100 has a rougher texture 104 than the remaining surface area 105 to create a stronger adherence to the top dressing layer 200 at the virtual hinge 106 that is created by lifting the top dressing component 200. This rougher-textured hinged end 103, 104 can be indicated to the user in various ways, by visible differences in the texture, by a pattern or symbol printed on the bandage or omission of same, or by other means.

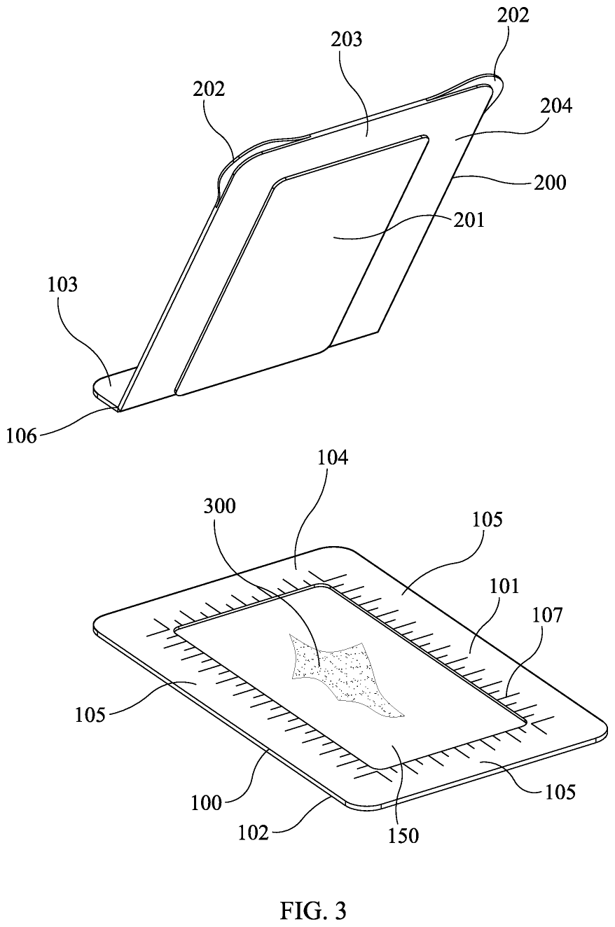

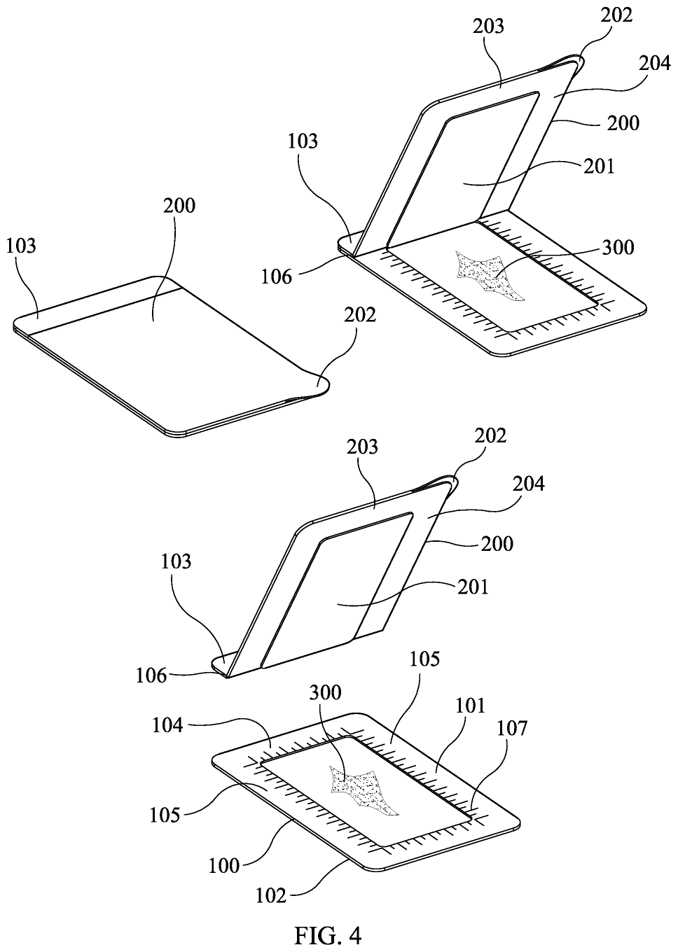

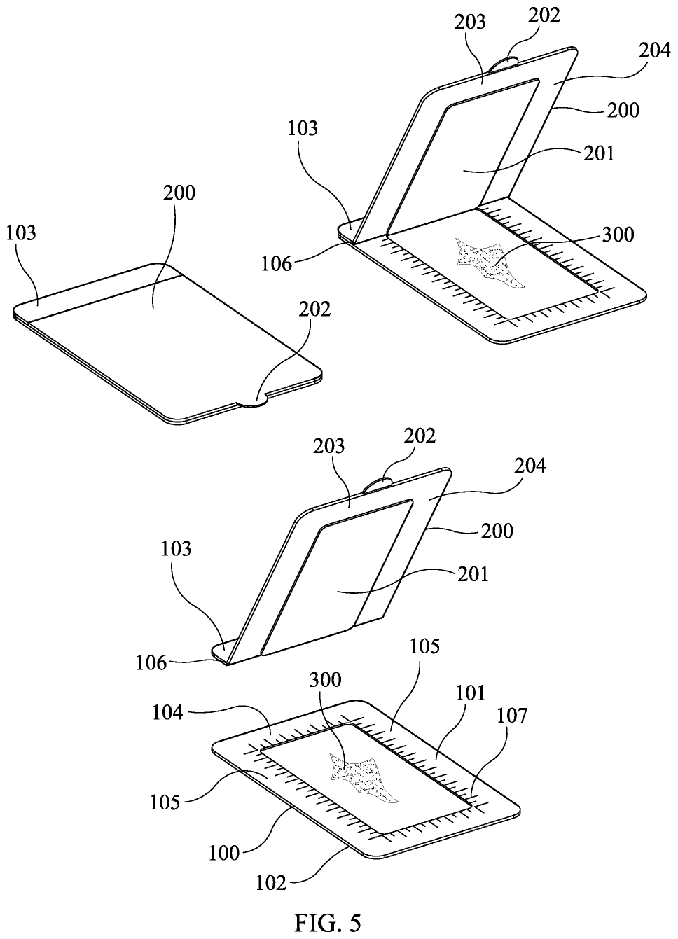

The top dressing layer 200 has a wound-facing surface 203 having a dressing or other medicated layer 201. Any type of dressing known in the art can be utilized. A portion of the wound-facing surface 203 has an adhesive layer 204 along its border that allows the top dressing layer to be affixed to the base border layer 100. The top dressing layer 200 further has at least one lift tab 202, being a curved tab protruding from the end or from at least one corner opposite the virtual hinged end 103 of the dressing or on the corners opposite the hinged end. The lift tab 202 portion of the top dressing layer 200 does not have any adhesive. It should be noted that although the inventor's preferred embodiment of the lift tabs 202 is shown in FIGS. 1-5, other shapes or configurations of tab placement along the top layer are contemplated by this invention. The number of lift tabs may be dependent on the total size and dimensions of the bandage layers 100, 200.

Product testing has shown that the material for the base border layer 100 must create an adhesive force between itself and the top dressing layer 200 that is strong enough to keep the window bandage sealed while enabling the top dressing layer 200 to be lifted without inadvertently lifting the entire bandage. The adhesive force at the hinge should be equal to or slightly less than the adhesive force between the skin and the bottom border layer 100. Additionally, the top dressing layer 200 can be made smaller by approximately 2 mm to 3 mm compared to the base border layer 100. This allows some give in the placement of the top layer so that it does not have to match perfectly with the dimensions of the base border layer component. The base border layer 100 can also feature hold-down tabs (not shown in the figures) to assist with lifting the top dressing layer.

In its closed state as shown in FIG. 1, the window bandage completely covers and protects the wound site 300. The adhesive component affixing the base border layer and the top dressing layer creates a seal strong and secure enough to not expose the wound. When the top dressing layer 200 is lifted by the lift tabs 202, it swings open and remains affixed to the bottom border layer 100 on the hinged end 103 of the base border layer. Because the hinged end 103 of base border has a rougher texture 104 than the remaining smoother sides of the base 105, the adhesive force keeps the top dressing layer affixed on that one hinged side. In this open state shown in FIG. 2, the patient's wound 300 is exposed through the window opening 150 of the base border component 100 of the bandage allowing treatment to be administered as needed. Should the top dressing layer 200 need to be changed, it can be removed completely from the base 100 by applying slightly stronger force and pulling it off the hinged end 103. A replacement top dressing layer 200 can be affixed to the same base border layer 100. The present invention thus resolves the need to remove bandages completely and risk abrasion or other secondary injuries to the patient's skin while also providing time savings to medical personnel or other wound caregivers.

It is to be understood that while a preferred embodiment of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification and drawings.

1. A hinged window bandage comprising:
A base border support layer surrounding an exposed window opening, said base border support layer having an upper surface and a bottom surface;
said bottom surface having an adhesive layer contacting the skin;
a top dressing layer having a wound-facing surface with an adhesive layer along its border, a central wound dressing area and having at least one lift tab along one end, and a hinged end opposite the lift tab end;
said wound dressing further having a closed position and an open position;
a section along one end of said upper surface of the base border support layer having a rougher texture than the remaining surface area of the upper surface and having stronger adhesive properties allowing the top dressing layer to be lifted in a hinged manner while remaining adhered to the rougher-textured section of said upper surface;
whereby the hinged window bandage completely covers and protects a wound in a closed position, and whereby pulling up on the lift tab creates a hinged opening between the underside of the top dressing layer and the base border layer, allowing access to the wound without the need to remove the hinged window bandage completely from the skin.

2. The top dressing layer of claim 1 being partially removeable by lifting the top dressing layer by the lift tabs.

3. The top dressing layer of claim 1 being completely removeable and replaceable with a new top dressing layer by applying additional force to peel off the top dressing layer from the base border layer.

4. The upper surface of the base border support layer of claim 1 having one end with a rougher texture than that of the surface area of the rest of the upper surface, creating greater adherence between the base border layer and the top dressing layer.

5. The base border support layer of claim 1 having a pattern of measurement markings printed along the edge of said upper surface of the base border support layer along the exposed window opening.

6. The hinged window bandage of claim 1 wherein the top dressing layer has the same length and width dimensions as the base border support layer.

7. The hinged window bandage of claim 1 wherein the top dressing layer is between 2 mm and 3 mm shorter in length and width compared to the base border layer.

8. The hinged window bandage of claim 1 wherein the lift tabs are curved protrusions located on the corners of the top dressing layer, opposite to the hinged side of the wound dressing, said lift tabs further having no adhesive layer component.
